(19) United States
(12) Patent Application Publication
(10) Pub. No.: US 2023/0340401 A1
(43) Pub. Date: Oct. 26, 2023

(54) INTEGRATED SYSTEMS AND METHODS FOR COMBINING METHANOTROPHIC BACTERIAL BIOMASS PRODUCTION AND METHANATION PROCESS

(71) Applicant: Calysta, Inc., San Mateo, CA (US)

(72) Inventors: Alan SHAW, Menlo Park, CA (US); Lorraine Joan GIVER, Sunnyvale, CA (US)

(21) Appl. No.: 18/061,968

(22) Filed: Dec. 5, 2022

Related U.S. Application Data
(60) Provisional application No. 63/286,429, filed on Dec. 6, 2021.

(51) Int. Cl. C12N 1/30; C12P 5/02; C12M 1/107

(52) U.S. Cl. CPC C12N 1/30; C12P 5/023; C12M 21/04

(57) ABSTRACT

The present disclosure provides methods of combining methanotrophic bacterial biomass production with a methanation process, comprising: (a) culturing a methanotrophic bacterium in the presence of methane and oxygen to produce biomass and carbon dioxide; and (b) generating methane using the carbon dioxide produced in step (a) and hydrogen. Also provided are systems that comprise: (a) one or more bioreactors comprising a culture of a methanotrophic bacterium to produce biomass and carbon dioxide in the presence of methane, and (b) one or more reactors for generating methane, wherein the system is configured so that the carbon dioxide generated from reactor (a) is fed into reactor (b).

### BACKGROUND

Protein production by conventional agriculture based food supply chains is becoming a major issue in terms of global environmental pollution and land and water scarcity. At the same time, the demand for high quality protein products such as those having high percentage crude protein is on the increase globally. Growing demand for protein cannot be met sustainably by increasing meat and dairy production because of the low efficiency of converting feed to meat and dairy products. Plant-based protein sources, such as beans, are nutritionally valuable sources of protein, but require arable land and water, both of which will become limiting. New solutions, such as single cell protein (i.e., protein produced in microbial and algal cells), are being explored. Currently, microbial protein provides a relatively small proportion of human nutrition. Methanotrophic bacteria are a promising approach to single cell protein production, as they can use methane as their sole source of carbon. However, much of that carbon is lost as CO₂ in the process of fermentative growth. Carbon dioxide is a major contributor to global warming. Other single cell protein production systems and/or methods are needed that are more sustainable.

### BRIEF SUMMARY

The present disclosure provides methods of combining methanotrophic bacterial biomass production with a methanation process, comprising: (a) culturing a methanotrophic bacterium in the presence of methane and oxygen to produce biomass and carbon dioxide; and (b) generating methane using the carbon dioxide produced in step (a) and hydrogen. In some embodiments, the methane used in step (a) further comprises methane generated in step (b).

The present disclosure also provides a system that comprises: (a) one or more bioreactors comprising a culture of a methanotrophic bacterium to produce biomass and carbon dioxide in the presence of methane; and (b) one or more reactors for generating methane, wherein the system is configured so that the carbon dioxide generated from reactor (a) is fed into reactor (b).

### BRIEF DESCRIPTION OF THE SEVERAL VIEWS OF THE DRAWINGS

FIG. 1 depicts an exemplary schematic for an integrated system for coupling of methanotrophic biomass production with methanogenic biomethanation, wherein optionally, electrolysis is used to produce hydrogen used for biomethanation process and oxygen used for methanotrophic biomass production.

### DETAILED DESCRIPTION

The present disclosure provides integrated systems and methods for culturing methanotrophic bacteria to generate biomass combined with a biomethanation process comprising: (a) culturing a methanotrophic bacterium in the presence of methane and oxygen to produce biomass and carbon dioxide; and (b) generating methane using the carbon dioxide produced in step (a) and hydrogen. Such a method allows for carbon dioxide generated during methanotrophic biomass production to be converted to methane and fed back to the methanotrophic bacteria. Optionally, the oxygen used for step (a) or both the oxygen of step (a) and the hydrogen of step (b) are generated by electrolysis of water. Integrated systems and methods of the present disclosure have the advantage in sustainability over current methanotrophic biomass production methods and systems wherein CO₂ is released as waste gas.

Prior to setting forth this disclosure in more detail, it may be helpful to an understanding thereof to provide definitions of certain terms to be used herein. Additional definitions are set forth throughout this disclosure.

In the present description, the term “about” means+20% of the indicated range, value, or structure, unless otherwise indicated. The term “consisting essentially of” limits the scope of a claim to the specified materials or steps and those that do not materially affect the basic and novel characteristics of the claimed invention. It should be understood the terms “a” and “an” as used herein refer to “one or more” of the enumerated components. The use of the alternative (e.g., “or”) should be understood to mean either one, both, or any combination thereof of the alternatives. As used herein, the terms “include” and “have” are used synonymously, which terms and variants thereof are intended to be construed as non-limiting. The term “comprise” means the presence of the stated features, integers, steps, or components as referred to in the claims, but that it does not preclude the presence or addition of one or more other features, integers, steps, components, or groups thereof. Any ranges provided herein include all the values and narrower ranges in the ranges.

### I. Producing Biomass

The method of combining methanotrophic bacterial biomass production with a methanation process of the present disclosure comprises a step of culturing a methanotrophic bacterium in the presence of methane and oxygen to produce biomass and carbon dioxide.

A. Methanotrophic Bacterium

The methanotrophic bacterium that may be used in the methods of the present disclosure may be any methanotrophic bacterium having the ability to oxidize methane as a carbon and energy source.

Methanotrophic bacteria are classified into three groups based on their carbon assimilation pathways and internal membrane structure: type I (gamma proteobacteria), type II (alpha proteobacteria, and type X (gamma proteobacteria). Type I methanotrophs use the ribulose monophosphate (RuMP) pathway for carbon assimilation whereas type II methanotrophs use the serine pathway. Type X methanotrophs use the RuMP pathway but also express low levels of enzymes of the serine pathway.

Methanotrophic bacteria include obligate methanotrophs, which can only utilize C₁ substrates for carbon and energy sources, and facultative methanotrophs, which naturally have the ability to utilize some multi-carbon substrates as a carbon and energy source.

Exemplary facultative methanotrophs include some species of Methylocella, Methylocystis, and Methylocapsa (e.g., Methylocella silvestris, Methylocella palustris, Methylocella tundrae, Methylocystis daltona strain SB2, Methylocystis bryophila, and Methylocapsa aurea KYG), Methylobacterium organophilum (ATCC 27,886), Methylibium petroleiphilum, or high growth variants thereof.

Exemplary obligate methanotrophic bacteria include Methylococcus capsulatus Bath (NCIMB 11132), Methylomonas sp. 16a (ATCC PTA 2402), Methylosinus trichosporium OB3b (NRRL B-11,196), Methylosinus sporium (NRRL B-11,197), Methylocystis parvus (NRRL B-11,198), Methylomonas methanica (NRRL B-11,199), Methylomonas albus (NRRL B-11,200), Methylobacter capsulatus Y (NRRL B-11,201), Methylomonas flagellata sp. AJ-3670 (FERM P-2400), Methylacidiphilum infernorum and Methylomicrobium alcaliphilum, or high growth variants thereof.

In certain embodiments, the methanotrophic bacterium is selected from the group consisting of Methylomonas, Methylobacter, Methylococcus, Methylosinus, Methylocystis, Methylomicrobium, Methanomonas, and Methylocella

In certain embodiments, the methanotrophic bacterium is a methanotrophic bacterium that expresses soluble methane monooxygenase (sMMO). MMOs catalyze oxidation of methane to methanol in methanotrophic bacteria.

Preferably, the methanotrophic bacterium is Methylococcus, Methylocystis, Methylosinus, or Methylocella, including those that express sMMO.

In certain embodiments, the methanotrophic bacterium is Methylococcus capsulatus, including Methylococcus capsulatus Bath, Methylococcus capsulatus Texas, and Methylococcus capsulatus Aberdeen. Preferably, the methanotrophic bacterium is Methylococcus capsulatus Bath. It is a thermophilic bacterium with an optimum growth temperature at about 45° C. M. capsulatus Bath is a Type I methanotroph.

B. Culturing Methanotrophic Bacterium

Methanotrophic bacteria may be grown by continuous culture methodologies in a controlled culture unit, such as a fermenter, bioreactor, hollow fiber cell, or the like. Continuous culture systems are systems where a defined culture medium (or its component(s)) is continuously added to a controlled culture unit while an equal amount of used (“conditioned”) media is removed simultaneously for processing. Continuous culture systems generally maintain the cells at a constant high, liquid phase density where cells are primarily in logarithmic growth phase.

A continuous culture system allows for the modulation of one or more factors that affect cell growth or end product concentration. For example, one method may maintain a limited nutrient at a fixed rate (e.g., carbon source, nitrogen) and allow one or more other parameters to change over time. In certain embodiments, several factors affecting growth may be continuously altered while cell concentration, as measured by media turbidity, is kept constant. The goal of a continuous culture system is to maintain steady state growth conditions while balancing cell loss due to media being drawn off against the cell growth rate. Methods of modulating nutrients and growth factors for continuous culture processes and techniques for maximizing the rate of product formation are well known in the art (see, e.g., Thomas D. Brock, Biotechnology: A Textbook of Industrial Microbiology, 2^{nd} Ed. (1989) Sinauer Associates, Inc., Sunderland, M A; Deshpande, Appl. Biochem. Biotechnol. 36:227, 1992).

In certain embodiments, the culture is in the presence of a carbon substrate as a source of energy for a methanotrophic bacterium. The carbon substrate may be methane only or may be methane in combination of one or more additional carbon substrates. Suitable additional substrates include C₁ substrates, such as methanol, formaldehyde, formic acid (formate), carbon monoxide, carbon dioxide, methylated amines (methylamine, dimethylamine, trimethylamine, etc.), methylated thiols, or methyl halogens (bromomethane, chloromethane, iodomethane, dichloromethane, etc.). In certain embodiments, the carbon substrate comprises biogas, natural gas, unconventional natural gas, methane off-gas from methanogenic culture, or a combination thereof.

“Biogas” typically refers to a mixture of gases produced by the biological breakdown of organic matter, e.g. biomass, in the absence of oxygen, such as biomass fermentation. Biogas is produced by anaerobic digestion or fermentation of biodegradable materials, i.e. of biomass, such as manure, sewage, municipal waste, green waste, plant material and energy crops. Thus, “biomass fermentation” denotes the anaerobic digestion or fermentation of biodegradable materials, i.e. of biomass. This type of biogas comprises primarily methane and carbon dioxide. Depending on the types of biodegradable materials, biogas may contain about 60-70 (vol %) of methane and about 30-40 (vol %), or may contain about 35-65 (vol %) of methane and about 15-50 (vol %) generated from anaerobic digestion of organic materials in landfills. Biogas may also include small amounts of hydrogen sulphide, siloxanes, oxygen, nitrogen, ammonia, and/or some moisture. In certain embodiments, biogas used as a carbon substrate for culturing a methanotrophic bacterium is upgraded biogas resulting from removing one or more certain components, such as hydrogen sulphide, siloxanes, oxygen, nitrogen, ammonia, and/or moisture.

“Natural gas” (also called fossil gas) refers to a naturally occurring hydrocarbon gas mixture consisting of methane (about 80% to 98%) and commonly including varying amounts of other higher alkanes, and sometimes a small percentage of carbon dioxide, nitrogen, hydrogen sulfide, or helium.

“Unconventional natural gas” (also called unconventional gas) refers to natural gas obtained from sources of production that are in a given era and location considered to be new and different, such as coalbed methane, methane clathrate, shale gas, synthetic natural gas (e.g., oil shale gas), and tight gas.

In certain embodiments, a portion of the methane used in culturing a methanotrophic bacterium is generated by methanation (e.g., biomethanation) using carbon dioxide and hydrogen as substrates.

During bacterial culture, the pH of the fermentation mixtures will generally be regulated to be between about 6 and about 8, such as between about 6 and about 7, between about 7 and about 8, or between about 6.5 and about 7.5.

During bacterial culture, the temperature is maintained to be in the range optimal for the cultured bacterium. For example, for M. capsulatus Bath, the temperature may be between 40° C. and 45° C.

Preferably, the methanotrophic bacterium is M. capsulatus Bath. M. capsulatus Bath may be cultured using methane as its carbon source, air or pure oxygen for oxygenation, and ammonia as the nitrogen source. In certain embodiments, a carbon feedstock comprising methane used for culturing M. capsulatus is biogas (e.g., upgraded biogas or non-upgraded biogas), natural gas, unconventional natural gas, methane off-gas from methanogenic culture, or a combination thereof. In addition to these substrates, the bacterial culture will typically require water, phosphate, and several minerals such as magnesium, calcium, potassium, irons, copper, zinc, manganese, nickel, cobalt and molybdenum. Exemplary culture media include Higgins minimal nitrate salts medium (NSM) or MM-W1 medium, master mix feed (MMF), medium MMF1.1, medium MMS1.0, or AMS medium. The copper concentrations of these media may be adjusted as described herein.

A specified amount of copper element is typically provided by a corresponding or equivalent amount of a copper salt that contains the same number of mole of copper element. For example, 100 mg copper is about 1.57 mmol, and may be provided by about 394 mg CuSO₄·5H₂O.

The term “high copper conditions” refers to continuous culture conditions where the amount of copper in a continuous culture is more than 200 mg copper per kg dry cell weight (DCW). “Dry cell weight (DCW)” refers to the dry weight of biomass harvested from a bacterial culture.

Specified copper conditions are typically set up by controlling Cu feed rates in view of DCW harvest rates. For example, for a low copper (Cu) concentration of 50 μg Cu/g of DCW (dry cell weight) and harvest rate 5 g/Uh of DCW, Cu (such as provided by CuSO₄·5H₂O) feed should be 250 μg Cu/Uh.

In certain embodiments, copper concentrations may be controlled by the use of a device (e.g., a pump) to feed a continuous culture at a defined rate.

In certain embodiments, the copper level under low copper conditions is from 1 to 100, from 1 to 10, from 10 to 20, from 20 to 30, from 30 to 40, from 40 to 50, from 50 to 60, from 60 to 70, from 70 to 80, from 80 to 90, from 90 to 100, from 1 to 90, from 1 to 80, from 1 to 70, from 1 to 60, from 1 to 50, from 1 to 40, from 1 to 30, from 10 to 90, from 10 to 80, from 10 to 70, from 10 to 60, from 10 to 50, from 10 to 40, from 10 to 30, from 20 to 90, preferably from 20 to 80, from 20 to 70, from 20 to 60, from 20 to 50, or from 20 to 40 mg copper/kg biomass. In certain other embodiments, a methanotrophic bacterium may be cultured under normal copper conditions. The term “normal copper conditions” refers to continuous culture conditions where the amount of copper in a continuous culture is in the range of 100 mg to 200 mg copper per kg dry cell weight (DCW). In certain embodiments, the copper level under normal copper conditions is from 100 to 180, from 100 to 170, from 100 to 160, from 100 to 150, from 100 to 140, from 100 to 130 mg copper/kg biomass.

In certain embodiments, a methanotrophic bacterium may be cultured under high copper conditions. The term “high copper conditions” refers to continuous culture conditions where the amount of copper in a continuous culture is more than 200 mg copper per kg dry cell weight (DCW). In certain embodiments, the copper level under high copper conditions is from 200 to 800, from 200 to 700, from 200 to 600, from 200 to 500, or from 200 to 400 mg copper/kg biomass.

The composition of medium MMS1.0 is as follows: 0.8 mM MgSO₄·7H₂O, 30 mM NaNO₃, 0.14 mM CaCl₂, 1.2 mM NaHCO₃, 2.35 mM KH₂PO₄, 3.4 mM K₂HPO₄, 20.7 μM Na₂MoO₄·2H₂O, 6 μM CuSO₄·5H₂O, 10 μM Fe^{III}—Na-EDTA, and 1 mL per liter of a trace metals solution (containing per liter: 500 mg FeSO₄·7H₂O, 400 mg ZnSO₄·7H₂O, 20 mg MnCl₂·7H₂O, 50 mg CoCl₂·6H₂O, 10 mg NiCl₂·6H₂O, 15 mg H₃BO₃, 250 mg EDTA). The final pH of the media is 7.0±0.1.

The AMS medium contains the following per liter: 10 mg NH₃, 75 mg H₃PO₄·2H₂O, 380 mg MgSO₄·7H₂O, 100 mg CaCl₂·2H₂O, 200 mg K₂SO₄, 75 mg FeSO₄·7H₂O, 1.0 mg CuSO₄·5H₂O, 0.96 mg ZnSO₄·7H₂O, 120 μg CoCl₂·6H₂O, 48 μg MnCl₂·4H₂O, 36 μg H₃BO₃, 24 μg NiCl₂·6H₂O and 1.20 μg NaMoO₄·2H₂O.

The composition of medium MMF1.1 is as follows: 0.8 mM MgSO₄·7H₂O, 40 mM NaNO₃, 0.14 mM CaCl₂, 6 mM NaHCO₃, 4.7 mM KH₂PO₄, 6.8 mM K₂HPO₄, 20.7 μM Na2MoO₄·2H₂O, 6 μM CuSO₄·5H₂O, 10 μM Fe^{III}—Na-EDTA, and 1 mL per liter of trace metals solution (containing, per liter 500 mg FeSO₄·7H₂O, 400 mg ZnSO₄·7H₂O, 20 mg MnCl₂·7H₂O, 50 mg CoCl₂·6H₂O, 10 mg NiCl₂·6H₂O, 15 mg H₃BO₃, 250 mg EDTA).

Suitable fermenters for culturing methanotrophic bacteria may be of the loop-type or air-lift reactors. Exemplary fermenters include U-loop fermenters (see U.S. Pat. No. 7,579,163, WO2017/218978), serpentine fermenters (see WO 2018/132379), and Kylindros fermenters (see WO 2019/036372).

In certain embodiments, the methanotrophic bacterium is cultured under good manufacturing practice (GMP) conditions. As used herein, the term “good manufacturing practice” or “GMP” refers to regulations promulgated by the US Food and Drug Administration under the authority of the Federal Food, Drug, and Cosmetic Act in 21 CFR 110 (for human food) and 111 (for dietary supplements) or comparable regulations set forth in jurisdictions outside the U.S that describe conditions and practices that are necessary for the manufacturing, processing, packing or storage of food to ensure its safety and wholesomeness.

In certain embodiments, the methanotrophic bacterium is cultured as an isolated culture without the presence of another organism. In certain other embodiments, the methanotrophic bacterium may be grown with one or more heterologous organisms (e.g., one or more heterologous bacteria) that may aid with growth of the methanotrophic bacterium. For example, a methanotrophic bacterium (e.g., Methylococcus capsulatus Bath) may be cultured with Cupriavidus sp., Aneurinibacillus danicus, or both and optionally in combination with Brevibacillus agri.

C. Bacterial Biomass

The term “bacterial biomass” refers to organic material collected from bacterial culture. Bacterial biomass primarily (i.e., more than 50% w/w) comprises bacterial cells, but may include other materials such as lysed bacterial cells, bacterial cell membranes, inclusion bodies, and extracellular material (e.g., products secreted or excreted into the culture medium), or any combination thereof that are collected from bacterial fermentation along with bacterial cells. Preferably, the biomass includes more than 60%, 70%, 75%, 80%, 85%, 90% or 95% cells collected from bacterial fermentation.

Bacterial biomass may be harvested from bacterial culture by various techniques, such as sedimentation, microfiltration, ultrafiltration, spray drying. Preferably, biomass is harvested from bacterial culture by centrifugation (e.g., at 4,000×g for 10 minutes at 10° C. For example, a fermentation broth (cells and liquid) may be collected and centrifuged. After centrifugation, the liquid can be discarded, and the precipitated cells may be saved and optionally lyophilized.

In certain embodiments, the biomass is processed by one or more additional steps to obtain a biomass derivative. As used herein the term “derivative” when used in relation to a biomass, includes any product which may be derived from such a material using a downstream processing technique or techniques known in the art, such as separation of a biomass material from a fermentation medium or liquid by centrifugation and/or filtration methods; homogenization or cell disruption by use of high pressure homogenizers or bead mills or sonication; digestion or lysis of the cells and their components by activation of endogenous enzymes or additions or external enzymes; various heat treatments; and drying by evaporation, spray drying, drum drying or freeze drying. Biomass derivatives include biomass autolysates, biomass lysates, biomass extracts, biomass isolates, biomass suspension, biomass homogenates, and biomass digestates (also referred to as “digests”). The biomass product may be in the form of a flowable aqueous paste, a slurry or a dried powder.

A “biomass lysate” refers to a biomass of which cells that have been lysed (i.e., the cell wall and/or membrane of the cells have been broken down). The cell lysis may be performed for example by electrochemical lysis (e.g., using hydroxide ions that are created electrochemically within the device by a palladium electrode, porating the membrane of a cell causing cell lysis), chemical lysis (e.g., by chemically solubilizing proteins and lipids within cell membrane), acoustic lysis (e.g., using ultrasonic waves to generate high and low pressure that causes cavitation and in turn cell lysis), or mechanical lysis (e.g., using physical penetration to break cell membrane).

A “biomass digestate” refers to one or more components of a biomass that have been enzymatically processed. Examples of biomass digestates include autolysates and hydrolysates, which are formed by autolysis or hydrolysis, respectively. Digestion of the biomass, such as by autolysis or hydrolysis, allows for the production of free amino acids and short-chain peptides.

A “biomass hydrolysate” refers to a biomass that has undergone digestion by enzymes exogenously supplied to the biomass.

A “biomass autolysate” refers to a biomass derivative that has undergone a digestion by enzymes naturally present in the biomass, known as autolysis. In some cases, additional exogenous enzymes (e.g. proteases, lipases, catalases) can be added to the biomass to enhance or accelerate the autolysis process. It will generally be conducted by incubation of the bacterial culture under carefully controlled conditions. Autolysis of the biomass may be performed by concentrating a culture of the methanotrophic bacterium and warming the concentrated culture to a temperature of about 50-60° C., for a period of time sufficient to produce an autolysate. Following autolysis, the autolysate may be heat inactivated by at a temperature of about 70-80° C., and then a soluble fraction of the autolysate, which includes free amino acids, may be isolated. In some embodiments, an autolysate is produced by 1) fermentation of the methanotrophic bacterium, (2) concentration of the fermentation product by centrifugation, filtration or evaporation, (3) homogenization, (4) autolysis with or without enzyme addition, (5) pasteurization, and (6) spray drying.

A “biomass extract” refers to a biomass component that has been separated from other components of the biomass. Other extracts could be enriched in specific recombinant proteins expressed in the C1 biomass (e.g. animal growth factors).

A “biomass isolate” refers to a biomass component that has been separated and purified. For example, for some growth media applications, it may be important to separate the soluble fraction from the residual particulate cell walls and cell debris, leading to a more soluble isolate and a particulate product. Examples of biomass isolates that may be used included filtrate and purified extracts, a soluble fraction or an insoluble fraction.

A “biomass suspension” refers to a mixture including biomass cells suspended in a liquid medium.

A “biomass homogenate” refers to a biomass that has been homogenized to release the contents of the cell. Homogenization of the biomass may be performed by sonication, bead homogenization, freeze/thaw cycles, with a Dounce homogenizer, or mortar and pestle. A biomass homogenate may be or include a viscous protein slurry containing both soluble and particulate cellular components.

In certain embodiments, bacterial biomass consists essentially of or consists of the biomass harvested from a methanotrophic bacterium.

In certain embodiments, the biomass of methanotrophic bacterium has at least 60% crude protein, such as at least 61%, 62%, 63%, 64%, 65%, 66%, 67%, 68%, 69%, 70%, 71%, 72%, 73%, 74%, 75%, 76%, 77%, 78%, 79%, 80%, or 81% crude protein. “Crude protein,” “crude protein content,” “crude protein concentration,” or “percentage crude protein” is a measurement of nitrogen in a protein sample. The amount of nitrogen is indicative of the amount of protein in the sample. The crude protein content of biomass or protein isolate disclosed herein is measured by the Dumas method. In certain embodiments, the bacterial biomass and/or the biomass of methanotrophic bacterium is composed of about 60% to about 99%, about 65% to about 99%, about 71% to about 99%, about 75% to about 99%, about 80% to about 99%, 82% to about 99%, about 60% to about 95%, about 65% to about 95%, about 71% to about 95%, about 75% to about 95%, about 80% to about 95%, about 82% to about 95%, about 60% to about 90%, about 65% to about 90%, about 71% to about 90%, about 75% to about 90%, about 80% to about 90%, about 82% to about 90%, about 60% to about 85%, about 65% to about 85%, about 71% to about 85%, about 75% to about 85%, about 60% to about 80%, about 65% to about 80%, or about 71% to about 80%, crude protein.

In certain embodiments, the bacterial biomass and/or the biomass of methanotrophic bacterium has at least 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, or 80% true protein “True protein,” “true protein content,” “true protein concentration,” or “percentage true protein” is a measurement of crude protein minus the non-protein nitrogen content in a protein sample. In certain embodiments, the bacterial biomass and/or the biomass of methanotrophic bacterium is composed of about 60% to about 99%, about 65% to about 99%, about 70% to about 99%, about 75% to about 99%, about 80% to about 99%, about 60% to about 95%, about 65% to about 95%, about 70% to about 95%, about 75% to about 95%, about 80% to about 95%, about 60% to about 90%, about 65% to about 90%, about 70% to about 90%, about 75% to about 90%, about 80% to about 90%, about 60% to about 85%, about 65% to about 85%, about 70% to about 85%, about 75% to about 85%, about 60% to about 80%, about 65% to about 80%, about 70% to about 80%, about 60% to about 75%, or about 60% to about 70% true protein.

In certain embodiments, the bacterial biomass and/or the biomass of methanotrophic bacterium has at most 14% such as at most 13%, 12%, or 11% ash. “Ash” is material left over in a sample that is burned (e.g., in furnace for 12-18 hours or overnight at 550° C.).

In certain embodiments, the bacterial biomass and/or the biomass of methanotrophic bacterium has at most 10%, such as at most 9%, 8%, 7%, 6%, or 5% nucleic acid. The nucleic acid content of biomass or protein isolate disclosed herein is measured using a Lucigen Masterpure Complete DNA & RNA Purification Kit MC85200.

In certain embodiments, the bacterial biomass and/or the biomass of methanotrophic bacterium cultured under normal or low copper conditions has at most 10%, 9%, 8%, 7.5%, 7%, 6%, or 5% crude fat. Crude fat may be measured by acid hydrolysis followed by organic solvent extraction. Briefly, fats or lipids in the bacterial biomass and/or the biomass of methanotrophic bacterium are first broken down via acid hydrolysis before being extracted via a solvent (e.g., ether or hexane). The solvent is then evaporated, and the material that remains is referred to “crude fat.”

In certain embodiments where a methanotrophic bacterium is cultured with one or more heterologous organisms, such as Methylococcus capsulatus Bath cultured with Cupriavidus sp., Aneurinibacillus danicus or both and optionally in combination with Brevibacillus agri, the bacterial biomass may comprise biomass from the heterologous organism(s) in addition to biomass from the methanotrophic bacterium.

Preferably, the bacterial biomass comprises primarily (i.e., more than 50%, such as more than 55%, more than 60%, more than 65%, more than 70%, more than 75%, more than 80%, more than 85% or more than 90% by weight) biomass from the methanotrophic bacterium.

In certain embodiments where a methanotrophic bacterium is cultured with one or more heterologous organisms, the bacterial biomass and/or the biomass of the methanotrophic bacterium has a copper level no more than 100 mg copper per kg DCW (mg/kg). In certain embodiments, the bacterial biomass and/or the biomass of the methanotrophic bacterium has a copper level from 1 to 100, from 1 to 10, from 10 to 20, from 20 to 30, from 30 to 40, from 40 to 50, from 50 to 60, from 60 to 70, from 70 to 80, from 80 to 90, from 90 to 100, from 1 to 90, from 1 to 80, from 1 to 70, from 1 to 60, from 1 to 50, from 1 to 40, from 1 to 30, from 10 to 90, from 10 to 80, from 10 to 70, from 10 to 60, from 10 to 50, from 10 to 40, from 10 to 30, from 20 to 90, from 20 to 80, from 20 to 70, from 20 to 60, from 20 to 50, or from 20 to 40 mg copper/kg DCW.

In certain embodiments where a methanotrophic bacterium is cultured with one or more heterologous organisms, the bacterial biomass and/or the biomass of the methanotrophic bacterium has at least 71%, 72%, 73%, 74%, 75%, 76%, 77%, 78%, 79%, 80%, or 81% crude protein, such as about 71% to about 99%, about 75% to about 99%, about 80% to about 99%, 82% to about 99%, about 71% to about 95%, about 75% to about 95%, about 80% to about 95%, about 82% to about 95%, about 71% to about 90%, about 75% to about 90%, about 80% to about 90%, about 82% to about 90%, about 71% to about 85%, about 75% to about 85% crude protein.

In certain embodiments where a methanotrophic bacterium is cultured with one or more heterologous organisms, the bacterial biomass and/or the biomass of methanotrophic bacterium has at least 60%, 61%, 62%, 63%, 64%, 65%, 66%, 67%, 68%, 69%, 70%, 71%, 72%, 73%, 74%, 75%, 76%, 77%, 78%, 79%, or 80% true protein, such as about 60% to about 99%, about 65% to about 99%, about 70% to about 99%, about 75% to about 99%, about 80% to about 99%, about 60% to about 95%, about 65% to about 95%, about 70% to about 95%, about 75% to about 95%, about 80% to about 95%, about 60% to about 90%, about 65% to about 90%, about 70% to about 90%, about 75% to about 90%, about 80% to about 90%, about 60% to about 85%, about 65% to about 85%, about 70% to about 85%, about 75% to about 85%, about 60% to about 80%, about 65% to about 80%, about 70% to about 80%, about 60% to about 75%, or about 60% to about 70% true protein.

In certain embodiments where a methanotrophic bacterium is cultured with one or more heterologous organisms, the bacterial biomass and/or the biomass of the methanotrophic bacterium has at most 14% such as at most 13%, 12%, or 11% ash.

In certain embodiments where a methanotrophic bacterium is cultured with one or more heterologous organisms, the bacterial biomass and/or the biomass of the methanotrophic bacterium has at most 10%, such as at most 9%, 8%, 7%, 7.5%, 6%, or 5% nucleic acid.

In certain embodiments where a methanotrophic bacterium is cultured with one or more heterologous organisms, the bacterial biomass and/or the biomass of methanotrophic bacterium cultured under low copper conditions has at most 10%, 9%, 8%, 7%, 6%, or 5% crude fat.

In certain embodiments, the biomass is harvested from a methanotrophic bacterium cultured under GMP conditions.

### II. Producing Methane

In addition to the step of culturing a methanotrophic bacterium in the presence of methane and oxygen to produce biomass and carbon dioxide described above, the method of combining methanotrophic bacterial biomass production with a methanation process of the present disclosure further comprises a step of generating methane using the produced carbon dioxide and hydrogen.

In certain embodiments, the methane used for culturing the methanotrophic bacterium further comprises methane generated in the step described in this section.

In certain embodiments, methane is produced using a chemical (or catalytic) process. The chemical process is called Sabatier reaction or Sabatier process. This reaction requires hydrogen and carbon dioxide, elevated temperatures (e.g., above 200° C.) and pressure (e.g., ranging from 5 to 100 bar), and may be accelerated by a metal catalyst, such as nickel or ruthenium on aluminum oxide.

In certain embodiments, methane is produced using a biological process (biomethanation) where carbon dioxide is converted to methane under aerobic conditions by a methanogenic microorganism.

A. Methanogenic Bacterium

In certain embodiments, the generating methane step comprises culturing a methanogenic microorganism. Methanogenic microorganisms, also referred to as methanogens, are capable of producing methane from carbon dioxide via a process called methanogenesis.

The methanogenic microorganism in the cell culture of the disclosure is obtainable from public collections of organisms, such as the American Type Culture Collection, the Deutsche Stammsammlung fur Mikroorganismen und Zellkulturen (DSMZ, Braunschweig, Germany), CBS (Utrecht, Netherlands) and the Oregon Collection of Methanogens, or they can be isolated from a variety of environmental sources. Such environmental sources include anaerobic soils and sands, bogs, swamps, marshes, estuaries, dense algal mats, both terrestrial and marine mud and sediments, deep ocean and deep well sites, sewage and organic waste sites and treatment facilities, animal intestinal tracts, volcano areas and feces. Suitable cell cultures may be pure (i.e., contain only cells of a single species) or may be mixed cultures (i.e., contain cells of more than one species). In certain embodiments, a pure cell culture of methanogenic microorganisms is used for the methods and systems of the disclosure.

In certain embodiments, the methanogenic microorganism is a methanogenic archaea. Methanogenic archaea include Methanothermobacter, Methanococcus, Methanomicrobium, Methanonatronarchaeia, Methanobrevibacter, Methanosarcina, Methanosaeta, and Methanopyrus. In certain embodiments, the methanogenic microorganism is Methanothermobacter.

In certain embodiments, the methanogenic microorganism is a hydrogenotrophic methanogenic microorganism. Hydrogenotrophic methanogenic microorganisms utilize hydrogen in the production of methane via a process called hydrogenotrophic methanogenesis. Hydrogenotrophic methanogenic microorganisms produce methane according to the following stoichiometric reaction:

4H₂+CO₂→CH₄+2H₂O

Exemplary methanogenic archaea for use in the methods and systems described herein include Methanobacterium alcaliphilum, Methanobacterium bryantii, Methanobacterium congolense, Methanobacterium defluvii, Methanobacterium espanolae, Methanobacterium formicicum, Methanobacterium ivanovii, Methanobacterium palustre, Methanobacterium thermaggregans, Methanobacterium uliginosum, Methanobrevibacter acididurans, Methanobrevibacter arbor iphilicus, Methanobrevibacter gottschalkii, Methanobrevibacter olleyae, Methanobrevibacter ruminantium, Methanobrevibacter smithii, Methanobrevibacter woesei, Methanobrevibacter wolinii, Methanothermobacter marburgensis (including Methanothermobacter marburgensis strain DSM 2133), Methanobacterium thermoautotrophicus, Methanothermobacter thermoflexus, Methanothermobacter thermophilus, Methanothermobacter wolfeii, Methanothermus sociabilis, Methanocorpusculum bavaricum, Methanocorpusculum parvum, Methanoculleus chikuoensis, Methanoculleus submarinus, Methanogenium frigidum, Methanogenium liminatans, Methanogenium marinum, Methanosarcina acetivorans, Methanosarcina barkeri, Methanosarcina mazei, Methanosarcina thermophila, Methanomicrobium mobile, Methanocaldococcus jannaschii, Methanococcus aeolicus, Methanococcus maripaludis, Methanococcus vannielii, Methanococcus voltaei, Methanothermococcus thermolithotrophicus, Methanopyrus kandleri, Methanosarcinia barkeri, Methanothermus fervidus, and Methanothermobacter thermoautotrophicus (including Methanothermobacter thermoautotrophicus strain UC 120910).

In certain embodiments, the methanogenic microorganism is Methanosarcinia barkeri, Methanococcus maripaludis, Methanothermobacter thermoautotrophicus, Methanobacterium thermoautotrophicus, or Methanothermobacter marburgensis.

B. Culturing Methanogenic Bacterium

Methanogenic microorganisms may be grown in a controlled culture unit, such as a fermenter, bioreactor, hollow fiber cell, a shake tank bioreactor, a continuous stirred tank bioreactor, an intermittent stirred tank bioreactor, a hollow fiber membrane bioreactor, a bubble column bioreactor, an internal-loop airlift bioreactor, an external-loop airlift bioreactor, a fluidized bed bioreactor, a packed bed bioreactor, a photo-bioreactor, a trickle bed reactor, a microbial electrolysis cell, and/or combinations thereof.

Methanogenic microorganisms may be grown as a batch process, fed-batch process or continuous culture.

In certain embodiments, the methanogenic microorganism is used as suspension with medium. The medium typically comprises at least a source of nitrogen, assimilable salts, a buffering agent and trace elements. Sulphur is provided to the cells by the reducing agent or may be provided extra, thus, the reducing agent and the sulphur-providing substance may or may not be the same. Sulphur may be provided to the cells by the provision of biogas. Prior to inoculation of the medium with the methanogenic microorganism, the medium may be degassed for being anaerobic for optimal methane production by the methanogenic microorganism. Techniques to prepare an oxygen free medium are known in the art, for example by flushing the medium with a gas mixture of 80% hydrogen and 20% carbon dioxide (v/v=volume per volume) or with nitrogen for 5 min per liter medium. Standard medium compositions may be taken from the literature and be adapted (Schoenheit, Moll et al, 1980; Schill, van Gulik et al, 1996; Liu, Schill et al, 1999). An example for a standard medium has the following composition (L⁻¹): 2.1 g NH₄Cl; 6.8 g KH₂PO₄; 3.4 g Na₂CO₃; 0.09 g Titriplex I; 0.04 g MgCl₂×6H₂O; 0.01 g FeCl₂×4H₂O; 0.2 mg CoCl₂×6H₂O; 1.2 mg NiCl₂×6H₂O; 0.2 mg NaMoO₄×2H₂O, the pH can be adjusted by titrating 1 M (NH₄)₂CO₃, NaOH or NH₄OH. The medium or single components of the medium are refreshed in a constant or stepwise mode during fermentation under continuous conditions. The feed rate or in-feed rate of medium or medium components is generally adjusted between 0.001 h⁻¹ and 0.1 h⁻¹. The out-flow rate corresponds to the in-flow rate plus water which is produced by the methanogenic microorganisms during methanogenesis. Said water can be re-used for various purposes, such as medium preparation.

The medium may be adjusted to the specific needs of the microorganism species, i.e. cell strain. In general, the fermentation conditions, i.e. medium composition, and other parameters, such as H₂/CO₂ partial pressure ratio, pH, temperature, stirring speed, pressure, oxidation reduction potential or medium or medium component (i.e. consumables) feed rate, i.e. fresh medium supply rate, have to be adjusted according to the specific needs of the microorganism strain selected and the procedural requirements depending for example on the phase of the methanogenic microorganisms in the reaction vessel.

A gas or gas mixture comprising the gases required for the production of methane, i.e. hydrogen and carbon dioxide, is fed into the methanation bioreactor and thus provided to the methanogenic microorganism. The gas feed may also comprise other gases which may be required for other purposes such as for example to adjust the oxidation reduction potential in the reaction vessel by addition of hydrogen sulfide or are required for other biological processes of the microorganisms or which are introduced as contaminants. This is especially the case if real gases are used as gas source for the method and systems of the disclosure. “Feed” means the introduction or transfer of a gas, liquid, suspension or any other substance into the into the reaction vessel or bioreactor of the disclosure. “Hydrogen gas feed” denotes the hydrogen comprising gas introduced into the reaction vessel or bioreactor comprising the methanogenic microorganism, and “carbon dioxide gas feed” denotes the carbon dioxide comprising gas introduced into the reaction vessel or bioreactor comprising the methanogenic microorganism. In certain embodiments, the hydrogen gas feed and/or carbon dioxide gas feed is pure according to general industrial standards. In certain embodiments, the hydrogen gas feed and/or carbon dioxide gas feed contains other gases, which may be contaminants. In certain embodiments, the methanogenic microorganism is cultured without other carbon sources (other than carbon dioxide gas feed).

In certain embodiments, the hydrogen gas feed is obtained from, wholly or in part, electrolysis of water. In certain embodiments, the carbon dioxide gas feed is obtained from, wholly or in part, the carbon dioxide off-gas produced by methanotrophic fermentation. In certain embodiments, the carbon dioxide gas feed further comprises waste CO₂ from other processes. The present method and system result in at least a partial consumption of carbon dioxide produced by the production of methanotrophic biomass, thereby reducing the amount of carbon dioxide released into the environment that would potentially contribute to global warming.

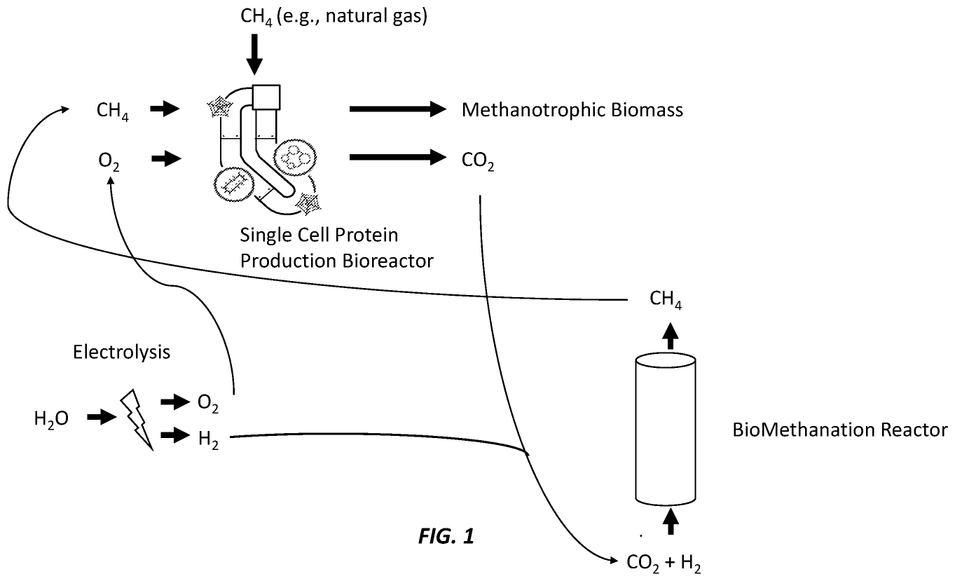

Methanogenic off-gas comprises methane produced by the methanogenic microorganisms. “Off-gas”, “exhaust gas”, “outgas” or “output gas” refers to the gaseous outcome of the reaction vessel or bioreactor of the disclosure, which is typically a gas mixture leaving the reaction vessel. The off-gas may also comprise water vapor and gases comprised in the in-gas, such as hydrogen, carbon dioxide, hydrogen sulfide. The off-gas mixture may further contain contaminants in the in-gas or originate from the cell culture, for example oxygen, compounds from biogas generation, and others. The qualitative and quantitative content of the off-gas depends on various factors, such as the phase of the methanogenic microorganisms in the reaction vessel, the total gas feed rate, and the composition of the gas feed. In certain embodiments, the methane content of the off-gas is at least 40%, 45%, 50%, 55%, 60%, 65%, 70%, 75%, 80%, 85%, 87%, 90%, 92%, 95% or 97%. During methanogenic cell growth phase, the methane content in the off-gas is typically between 50% and 80%. The methane from the off-gas may be separated by standard means, e.g. by membranes such as obtainable from Du Pont (Wilmington, DE, USA) or Gore (Newark, DE, USA). In certain embodiments, the methane off-gas is used as gas feed for culturing methanotrophic bacterium according to the methods and systems described herein. The present method and system result in supplementation of methane feedstock for methanotrophic fermentation from methane converted by methanogenic microorganisms from carbon dioxide gas produced during methanotrophic fermentation (see, FIG. 1).

In certain embodiments, the method of combining methanotrophic bacterial biomass production with a methanation process comprises a continuous cycle of: (a) culturing a methanotrophic bacterium in the presence of methane and oxygen to produce biomass and carbon dioxide; and (b) generating methane using the carbon dioxide produced in step (a) and hydrogen, wherein the methane generated in step (b) is used in step (a).

### III. Electrolysis

In certain embodiments, the oxygen of step (a), the hydrogen of step (b), or both the oxygen of step (a) and the hydrogen of step (b) are generated by electrolysis of water.

“Electrolysis” refers a method or process that uses an electric current to induce an otherwise non-spontaneous chemical reaction. In the process, an electric current passes through a substance, thereby causing a chemical change of said substance, usually the gaining or losing of electrons. In certain embodiments, electrolysis comprises an electrolytic cell, such as an electrolyser, e.g., Hofmann voltameter, composed of separated positive and negative electrodes (anode and cathode, respectively) immersed in an electrolyte solution containing ions or in a molten ionic compound. Reduction occurs at the cathode, where electrons are added that combine with positively charged cations in the solution. Oxidation occurs at the anode, where negatively charged anions give up electrons.

In certain embodiments, electrodes comprise noble metals, such as platinum. In certain embodiments, electrodes comprise inexpensive, non-corrosive materials. Electrodes for water electrolysis are generally known in the art and preferably comprise non-noble catalytic materials, for example, stainless steel, graphite, graphite-based materials, nickel, steel, a metal alloy or a metal oxide (e.g., titanium and/or iridium oxide). In certain embodiments, stainless steel and graphite are preferred.

Standard electrolyser can be obtained from various manufacturers such as from Hydrogen Technologies (Notodden/Porsgrunn, Norway), Proton Energy Systems (Wallingford, CT, USA), Heliocentris Energy Solutions AG (Berlin, Germany), Claind (Lenno, Italy), Hydrogenics GmbH (Gladbeck, Germany), Sylatech Analysentechnik GmbH (Walzbachtal, Germany), h-tec Wasserstoff-Energie-Systeme GmbH (Luebeck, Germany), zebotec GmbH (Konstanz, Germany), H₂ Logic (Herning, Denmark), QuinTech (Goeppingen, Germany), and electrolysis may be performed according to the manufacturer's instructions.

Energy for electrolysis of water may be provided by any energy source, such as renewable or non-renewable energy sources, such as electricity from combustion or gasification of fossil fuels, nuclear energy, wind power, solar power, geothermal power, hydro power, wave power, tidal power, biofuels, etc.

In the electrolysis of water, water is used as the substance through which the electronic current passes. The electronic current leads to the decomposition of the water into oxygen (O₂) and hydrogen (H₂). The overall reaction equation is:

2H₂O(liquid)→2H₂(gaseous)+O₂(gaseous).

Water for electrolysis may be obtained by any source, such as tap water, from rivers, lakes, sea water, rain, or waste water from industrial processes. In embodiments where the water does not have the necessary purity for the electrolysis, the water may be purified by distillation, filtration and/or centrifugation and other means, which are known to a person skilled in the art.

Electrolysis of water may occur directly in a reactor, in an external recirculation loop, or within an external electrolyzer.

Hydrogen gas produced from electrolysis, to be used as reducing power for biomethanation, may be pumped into a biomethanation reactor for the culture of a methanogenic microorganism. Oxygen gas produced from electrolysis may be pumped into a reactor for the culture of a methanotrophic bacterium.

### IV. Systems

In another aspect, the present disclosure provides a system that comprises: (a) one or more bioreactors comprising a culture of a methanotrophic bacterium to produce biomass and carbon dioxide in the presence of methane; and (b) one or more reactors for generating methane, wherein the system is configured so that the carbon dioxide generated from reactor (a) is fed into reactor (b).

In certain embodiments, the system comprises 1, 2, 3, 4, 5, 6, 7, 8, 9, 10, or more bioreactors comprising a culture of a methanotrophic bacterium. In certain embodiments, the system comprises 1, 2, 3, 4, 5, 6, 7, 8, 9, 10, or more reactors for generating methane.

Suitable methanotrophic bacteria, their carbon sources, and their culture conditions are described above in connection with methods of combining methanotrophic bacterial biomass production with a methanation process. In certain embodiment, the one or more reactors for generating methane are biomethanation reactor(s) comprising a culture a methanogenic microorganism. Suitable methanogenic microorganisms and their culture conditions are also described above in connection with methods of combining methanotrophic bacterial biomass production with methanation.

Liquid phase bioreactors (e.g., stirred tank, packed bed, one liquid phase, two liquid phase, hollow fiber membrane) are well known in the art and may be used for growth of microorganisms and biocatalysis.

By using gas phase bioreactors, substrates for bioproduction are absorbed from a gas by microorganisms, rather than from a liquid. Use of gas phase bioreactors with microorganisms is known in the art (see, e.g., U.S. Pat. Nos. 2,793,096; 4,999,302; 5,585,266; 5,079,168; and 6,143,556; U.S. Statutory Invention Registration H1430; U.S. Pat. Appl. Pub. No. US 2003/0032170; Emerging Technologies in Hazardous Waste Management III, 1993, eds. Tedder and Pohland, pp. 411-428, all of which are incorporated herein by reference). Exemplary gas phase bioreactors include single pass system, closed loop pumping system, and fluidized bed reactor. By utilizing gas phase bioreactors, methane or other gaseous substrates are readily available for bioconversion by polypeptides with, for example, monooxygenase activity.

Suitable methanation reactors for catalytic CO₂ methanation include fixed-bed reactors, structured reactors, fluidized-bed reactors, and slurry bubble column reactors. In certain embodiments, a fixed-bed reactor is used for large-scale catalytic CO₂ methanation. Heat management is an important factor for methanation reactors. Cooled fixed-bed reactors may be used to lower temperature in the reactor. In fluidized-bed reactors, gas flow introduced into the reactor fluidizes the catalyst particles and causes a high degree of mixing. This effect and the high heat capacity of the catalyst particles allows for nearly isothermal operation and the avoidance of temperature hotspots. In structured reactors, metallic structures are often part of the reactor interior or are used as catalyst carrier significantly enhancing the heat transfer from the catalyst to the cooling medium on the outer shell of the reactor tube. Slurry bubble column reactors include a liquid phase in the reactor, directly present on the catalyst surface where the heat of reaction is produced. Due to its high heat capacity and heat conductivity, this liquid facilitates heat management.

In certain embodiments, a methanation reactor for catalytic CO₂ methanation is a two-phase system. Exemplary two-phase reactors include structured reactors such as microchannel or honeycomb reactors. In certain embodiments, a methanation reactor for catalytic CO₂ methanation is a three-phase system. Fluidized-bed reactors can be operated with two or three phases, whereas slurry reactors are operated with three phases exclusively.

In certain embodiments, a methanation reactor is a honeycomb reactor. A honeycomb reactor is two-phase structured fixed-bed reactor that contains coated catalyst carriers made of stainless steel. It is designed as a multitube reactor, in which the metallic catalyst carriers are placed in parallel tubes. A honeycomb reactor is made of a combination of corrugated and plane metal sheets, which are jointly coiled up. The layers are form-fit pressed in a cladding tube. Typically, honeycomb structures are characterized by the number of parallel channels per square inch (CPSI). In certain embodiments, honeycomb structures of 100-600 CPSI are used corresponding to channel diameters of 0.1-2.8 mm. The feed gas flow enters the catalytically coated channels and if the reactor temperature is high enough (above 200° C.) the catalytic methanation reaction starts. CO₂ and H₂ are converted to CH₄ in the porous catalyst layer and reaction heat is released mostly at the channel inlets.

In certain embodiments, a methanation rector is a slurry bubble column reactor (SBCR). A slurry bubble column reactor may have three distinctive phases: a commercially available solid catalyst (particle size of 50-100 μm) suspended in a heat transfer liquid and fluidized by the educt gases (H₂ and CO₂).

Fermentors are generally defined as any vessel in which a fermentation process is carried out. Given the vast number of fermentation processes and the wide variety of fermentable substrates, fermentors can range from simple continuous stirred tank reactors found in the alcoholic beverage industry to highly complex, specialized vessels having gas distribution and internal structures tailored to a particular substrate and/or a particular biological species.

Fermentors useful in utilizing carbon-containing gases such as methane as a substrate for culturing single cell microorganisms such as bacteria which contain high proportions of proteins generally disperse a gas substrate containing a C₁ carbon compound within a liquid media containing one or more nutrients to provide a multi-phase mixture. This multi-phase mixture is contacted with one or more microbiological colonies that convert a portion of the C₁ carbon compound(s) in the gas substrate to proteins. The substrate composition, nutrients, and microbiological organisms comprising the colony (i.e., the biomass within the fermentor) can be variously adjusted or tailored to provide a desired final matrix of protein-containing biomass.

The growth phase and methane production phase of methanogenic microorganisms may occur in the same reaction vessel or occur in separate reaction vessels (cells grown in one reaction vessel and transferred to another reaction vessel for methane production). In certain embodiments, a single bioreactor is used. The bioreactor may comprise at least two reaction vessels, containing at least one for cell growth and at least one for methane production. These two reaction vessels may be linked, for example via tubes, pipes, etc/to transport the cell culture comprising the suspension of methanogenic microorganisms from one reaction vessel to the other or back if needed. Said two reaction vessels may also not be directly linked to each other, e.g., the methanogenic microorganism may be transferred from one reaction vessel to the other via another container.

Methanogenic microorganisms may also be cultured, for example, in a continuous stirred tank bioreactor, an intermittent stirred tank bioreactor, a hollow fiber membrane bioreactor, a bubble column bioreactor, an internal-loop airlift bioreactor, an external-loop airlift bioreactor, a fluidized bed bioreactor, a packed bed bioreactor, a photo-bioreactor, a trickle bed reactor, a microbial electrolysis cell, or any combination thereof, and operated in a batch, fed batch, continuous, semi-continuous, or perfusion mode. In batch mode (single batch), an initial amount of medium containing nutrients necessary for growth is added to the biological reactor, and the biological reactor is operated until the number of viable cells rises to a steady-state maximum, or stationary condition. In fed-batch mode, concentrated media or selected amounts of single nutrients are added at fixed intervals to the culture. Methanogenic microorganisms can survive for years under fed batch conditions, provided that any waste products are effectively minimized or removed to prevent loss of efficiency of methane production over time. Any inhibitory waste products may be removed by continuous perfusion production processes, well known in the art. Perfusion processes may involve simple dilution by continuous feeding of fresh medium into the culture, while the same volume is continuously withdrawn from the reactor. Perfusion processes may also involve continuous, selective removal of medium by centrifugation while cells are retained in the culture or by selective removal of toxic components by dialysis, adsorption, electrophoresis, or other methods. Continuously perfused cultures may be maintained for weeks, months or years.

Suitable bioreactors for methane production by methanogenic microorganisms may be a stratified bioreactor, cascaded bioreactor, an electro-biological reactor described in WO2011003081, and bioreactors described in WO2012110256. A stratified bioreactor has the carbon dioxide and hydrogen entering into the bottom of the bioreactor along with the nutrients for the bioreactor. A mechanical impeller is positioned on the top of the bioreactor and is used to move a mixing apparatus within the bioreactor. The bioreactor has three zones, A, B and C. Zone A at the bottom of the reactor is a high carbon dioxide zone. Zone B, in the middle of the bioreactor has a decreased carbon dioxide presence, and Zone C at the top end of the reactor has little if any carbon dioxide. The methane produced, and the spent medium is removed from the top of the bioreactor. In a cascaded bioreactor, the hydrogen, carbon dioxide and cell nutrients are fed into the bottom of a first compartment (A). In this compartment (A), even after processing, there is still a high level of carbon dioxide. The gas produced by the reaction in the first compartment (A) is then transferred from the top of the first compartment to the bottom of a second compartment (B) along with cell nutrients. In this second compartment (B), the carbon dioxide level is decreased from the levels found in the first compartment (A). The gas produced by the reaction in the second compartment (B) is transferred from the top of the second compartment (B) to the bottom of a third compartment (C) along with cell nutrients. In this third compartment (C), most (if not all) of the carbon dioxide has been removed and only the methane gas is left to be removed from the top of the compartment. In each of the compartments, spent medium can be removed from the compartments.

A fermenter may be sized relative to the volume of the CO₂ source. For example, a stream of 2,200,000 lb CO₂/day would require a CO₂ recovery/methane production fermentor of about 750,000 gal total capacity.

In certain embodiments, the fermenter substantially excludes oxygen to promote high levels of methane production. In certain embodiments, the hydrogen and/or carbon dioxide are fed through an oxygen scrubber prior to feeding into the fermenter for culturing a methanogenic microorganism.

Suitable fermenters for culturing methanotrophic bacteria may be of the loop-type or air-lift reactors. Exemplary fermenters include U-loop fermenters (see U.S. Pat. No. 7,579,163, WO2017/218978), serpentine fermenters (see WO 2018/132379), and Kylindros fermenters (see WO 2019/036372).

In certain embodiments, the methane in one or more bioreactors(s) of (a) comprises biogas, natural gas, unconventional natural gas.

In certain embodiments, the system is configured so that the methane generated from at least one or more reactors (b) is fed into at least one of the one or more bioreactors of (a).

In certain embodiments, the system further comprises (c) an electrolytic cell for hydrolyzing water to generate hydrogen and oxygen, wherein the system is configured so that hydrogen generated from the electrolytic cell (c) is fed into reactor (b), and wherein the oxygen generated from electrolytic cell (c) is fed into reactor (a).

In certain embodiments, the methanotrophic bacterium is an obligate methanotrophic bacterium.

In certain embodiments, the methanotrophic bacterium is Methylococcus capsulatus.

In certain embodiments, the methanotrophic bacterium is Methylococcus capsulatus Bath.

In certain embodiments, reactor (b) is a bioreactor comprising a culture of a methanogenic microorganism.

In certain embodiments, the methanogenic microorganism is a hydrogenotrophic methanogenic microorganism.

In certain embodiments, the hydrogenotrophic methanogenic microorganism is cultured without other carbon sources.

In certain embodiments, the methanogenic microorganism is Methanothermobacter.

In certain embodiments, the methanogenic microorganism is Methanothermobacter thermautotrophicus.

In certain embodiments, the methanogenic microorganism is Methanothermobacter thermautotrophicus strain UC 120910.

In certain embodiments, the methanogenic microorganism is Methanothermobacter marburgensis.

In certain embodiments, the methanogenic microorganism is Methanothermobacter marburgensis strain DSM 2133.

In certain embodiments, the methanogenic microorganism is Methanothermus fervidus.

In certain embodiments, of the methane that goes into a bioreactor of (a), about 50% may be converted into methanotrophic biomass. The remaining methane may be converted to carbon dioxide. The carbon dioxide from methanotrophic fermentation is recycled into reactor (b), which can in turn produce methane feedstock for methanotrophic fermentation. In certain embodiments, methanation in reactor (b) is insufficient to feed the bioreactor of (a) comprising a culture of methanotrophic bacterium, and methane from another source (e.g., biogas or natural gas) is additionally fed to the bioreactor of (b).

In certain embodiments, ½, ⅔, or ¾ of the bioreactors of (a) are fed with natural gas, biogas or unconventional natural gas. The CO₂ generated from those bioreactors are fed into one or more bioreactors (b) to be converted into methane by methanogenic micoorganisms. The methane produced in reactor(s) (b) may be used to feed the remaining reactors of (a). In an exemplary system, the system has 6 bioreactors in (a), 4 of which are fed natural gas. The CO₂ generated from those 4 bioreactors is fed into the reactor(s) of (b) to be converted into methane by a methanogenic microorganism, and the renewed methane is fed into the remaining 2 bioreactors of (a). Thus, ⅓ of the biomass produced in this exemplary system would be ‘green’ in that it is made from renewable methane.

The various embodiments described above can be combined to provide further embodiments. All of the U.S. patents, U.S. patent application publications, U.S. patent applications, foreign patents, foreign patent applications and non-patent publications referred to in this specification and/or listed in the Application Data Sheet, including U.S. Provisional Patent Application No. 63/286,429, filed on Dec. 6, 2021, are incorporated herein by reference, in their entirety. Aspects of the embodiments can be modified, if necessary to employ concepts of the various patents, applications and publications to provide yet further embodiments.

These and other changes can be made to the embodiments in light of the above-detailed description. In general, in the following claims, the terms used should not be construed to limit the claims to the specific embodiments disclosed in the specification and the claims, but should be construed to include all possible embodiments along with the full scope of equivalents to which such claims are entitled. Accordingly, the claims are not limited by the disclosure.

1. A method of combining methanotrophic bacterial biomass production with a methanation process, comprising:
(a) culturing a methanotrophic bacterium in the presence of methane and oxygen to produce biomass and carbon dioxide; and
(b) generating methane using the carbon dioxide produced in step (a) and hydrogen.

2. The method of claim 1, wherein the methane used in step (a) comprises biogas or natural gas.

3. The method of claim 1, wherein the methane used in step (a) further comprises methane generated in step (b).

4. The method of claim 3, wherein the method comprises a continuous cycle of steps (a) and (b).

5. The method of claim 1, wherein the methanotrophic bacterium is:
(a) an obligate methanotrophic bacterium;
(b) a facultative methanotrophic bacterium;
(c) selected from the group consisting of Methylomonas, Methylobacter, Methylococcus, Methylosinus, Methylocystis, Methylomicrobium, Methanomonas, and Methylocella;
(d) Methylococcus capsulatus: or
(e) Methylococcus capsulatus Bath.

6-9. (canceled)

10. The method of claim 1, wherein step (b) comprises culturing a methanogenic microorganism.

11. The method of claim 10, wherein the methanogenic microorganism is
(a) a hydrogenotrophic methanogenic microorganism;
(b) Methanothermobacter, Methanococcus, Methanomicrobium, Methanonatronarchaeia, Methanobrevibacter, Methanosarcina, Methanosaeta, Methanopyrus, or Methanothermobacter; or
(c) Methanosarcinia barkeri, Methanococcus maripaludis, Methanothermobacter thermautotrophicus, Methanothermobacter thermautotrophicus strain UC 120910, Methanothermobacter marburgensis, Methanothermobacter marburgensis strain DSM 2133, or Methanothermus fervidus.

12. The method of claim 11, wherein the culturing the methanogenic microorganism in step (b) is without other carbon sources.

13-20. (canceled)

21. The method of claim 1, wherein the oxygen of step (a), the hydrogen of step (b), or both the oxygen of step (a) and the hydrogen of step (b) are generated by electrolysis of water.

22. The method of claim 1, further comprising:
(c) processing the biomass of step (a) to produce a biomass homogenate, a biomass extract, a biomass lysate, a biomass autolysate, or a biomass digestate.

23. A system that comprises:
(a) one or more bioreactors comprising a culture of a methanotrophic bacterium to produce biomass and carbon dioxide in the presence of methane, and
(b) one or more reactors for generating methane,
wherein the system is configured so that the carbon dioxide generated from reactor (a) is fed into reactor (b).

24. The system of claim 23, wherein the methane in at least one of the one or more bioreactor(s) (a) comprises biogas or natural gas.

25. The system of claim 23, wherein the system is configured so that the methane generated from at least one of the one or more reactors (b) is fed into at least one of the one or more bioreactor (a).

26. The system of claim 23, further comprising (c) an electrolytic cell for hydrolyzing water to generate hydrogen and oxygen, wherein the system is configured so that the hydrogen generated from electrolytic cell (c) is fed into reactor (b), and wherein the oxygen generated from electrolytic cell (c) is fed into reactor (a).

27. The system of claim 23, wherein the methanotrophic bacterium is an obligate methanotrophic bacterium, Methylococcus capsulatus, or Methylococcus capsulatus Bath.

28. (canceled)

29. (canceled)

30. The system of claim 23, wherein reactor (b) is a bioreactor comprising a culture of a methanogenic microorganism.

31. The system of claim 30, wherein the methanogenic microorganism is a hydrogenotrophic methanogenic microorganism.

32. The system of claim 31, wherein the hydrogenotrophic methanogenic microorganism is cultured without other carbon sources.

33. The system of claim 32, wherein the methanogenic microorganism is Methanothermobacter, Methanothermobacter thermautotrophicus, Methanothermobacter thermautotrophicus UC 120910, Methanothermobacter marburgensis, Melhanolhermobacler marburgensis DSM 2133, or Methanothermus fervidus.

34-38. (canceled)
